(19) United States
(12) Patent Application Publication
(10) Pub. No.: US 2017/0333131 A1
(43) Pub. Date: Nov. 23, 2017

(54) A SYSTEM AND METHOD FOR TREATING ARTERY

(71) Applicant: Ajoy I. SINGH, Mumbai, Maharashtra (IN)

(72) Inventor: Ajoy I. SINGH, Mumbai, Maharashtra (IN)

(21) Appl. No.: 15/540,736

(22) PCT Filed: Dec. 23, 2015
(86) PCT No.: PCT/IN2015/050212

§ 371 Date: Jun. 29, 2017

(30) Foreign Application Priority Data
Dec. 29, 2014 (IN) 4201/MUM/2014

(51) Int. Cl. A61B 18/24; A61B 17/3207; A61B 1/05; A61B 1/045; A61B 18/22; A61B 17/00; A61B 18/00

(52) U.S. Cl. CPC A61B 18/245; A61B 1/05; A61B 17/3207; A61B 1/045; A61B 2017/00017; A61B 2018/00577; A61B 2018/00982; A61B 2018/2277; A61B 2017/320733

(57) ABSTRACT

The present invention provides a system and method for treating artery having atherosclerosis. The system comprises a flexible device for wrapping around the diseased artery, at least an imaging fiber and/or an ablation fiber removably adapted on the inner side of the flexible device for imaging and/or treating the said artery and a controller for controlling the process of imaging or treatment on the said artery.

### FIELD OF THE INVENTION

The present invention relates to a system for treating a diseased artery. Particularly, the present invention relates to a system for treating a diseased artery having atherosclerosis and a method of treating the diseased artery.

### BACKGROUND ART OF THE INVENTION

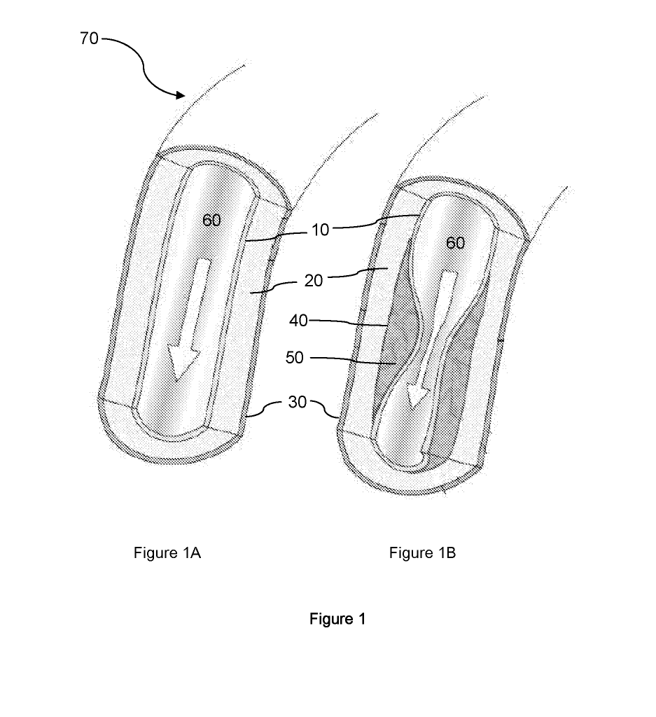

It is known that a normal artery (70) as shown in FIG. 1A has a lumen (60) defined by a three layered structure, namely innermost layer called as tunica intima (10), a middle layer called as tunica media (20) and an outermost layer called as tunica adventitia (30). Atherosclerosis is a disorder of arteries that afflicts a large proportion of humanity. The disease is characterized by the blockages in arteries develop due to accumulation of cholesterol, fats and other chemicals between the tunica intima (10) and the tunica media (20) of the diseased artery wall as shown in FIG. 1B. This accumulation is enveloped in a thin fibrous capsule (40). The fibrous capsule (12) with the accumulations of cholesterol, fats and other chemicals are often collectively referred to as “atherosclerotic or atheromatous plaque” (50). This plaque (50) also contains cells of various types, predominantly macrophages, giant cells and smooth muscle cells. These cells are a consequence of the inflammatory nature of the disease process afflicts the wall of the diseased artery. Thus, atherosclerosis is characterized by enlargement of the diseased artery wall as shown in FIG. 1B. Principally restriction is due to atherosclerotic plaque bulging into the inner space (lumen) of the diseased artery. This leads to increased arterial wall stiffness and reduced internal diameter of the diseased artery, and consequently reduced blood flow through the diseased artery. If these contents are released to the inside of the vessel wall it may lead to a heart attack or a brain stroke due to thrombosis (clotting of blood inside the diseased artery). The degree of atherosclerosis is measured as an increase in Intima Media Thickness (“IMT”).

Further, it has since been recognized that atherosclerosis is a systemic disease and blockages (stenoses) a local manifestation of the disease process which may also widespread and distributed over a region of the arterial tree as shown in FIG. 1C. The disease manifests itself in an angiogram as a local narrowing/blockage, but pathology studies confirm the existence of disease in areas that are not narrowed as well.

In real life, the biological process accompanying atherosclerosis is a lot more complex, including a self-healing mechanism of the human or animal body that attempts to minimize the constriction of the diseased artery, called stenosis in medical terminology. The self-healing mechanism functions by “remodeling” the diseased artery. The constituents of these prolonged depositions, called atheroma, include macrophage cells, cellular debris, dead cells and living cells, as well as the fibrous tissue covering of the atheroma itself. Over time, calcification can also occur between the atheroma layer and the underlying smooth muscle cell layer of the vessel wall.

Numerous medical equipments and techniques are available today for unblocking coronary arteries blocked by the atheromatous plaque. Chief among them are balloon angioplasty, Stents, rotational atherectomy, directional atherectomy and transluminal extraction atherectomy.

During an actual medical procedure, each of above techniques typically uses the catheter. For guidance, a guide wire is typically inserted first before the catheter. The catheter is then passed through the diseased artery over the guide wire to reach the target area. The approach is through the diseased artery lumen. All these procedures may cause some degree of injury to the innermost lining of the tunica intima. Hence, many of these procedures have a high rate of re-blockage due to cellular proliferation, which follows any injury to the tunica intima. The advent of drug eluting stents has reduced this incidence significantly, but limitations remain.

Treatment of atherosclerosis by all known present technologies is carried out or suggested to be carried out only when blockage of the vessel is more than 50% of internal diameter of the diseased artery as the said technologies are nearly ineffective in treating early and about mid-stage plaque formation. This is particularly troublesome in view of the fact that mid-stage, vulnerable plaque formation with minimum lumen intrusion is now clinically considered to be even more dangerous owing to its tendency to rupture spontaneously, leading to immediate and severe heart attack or even instant death.

The U.S. Pat. No. 6,669,686 granted to the present inventor discloses a method that avoids approaching atherosclerosis through the diseased artery, more particularly, to a method for reducing the thickness of an arterial wall by ablation of the exterior of the diseased artery wall by laser ablating or removing the exterior layer of the arterial wall the tunica intima and inner layers of the tunica media are protected from damage. The flexibility of the diseased artery is improved due to the reduced effective wall thickness after ablation thus relieving stenosis and improving blood flow through the diseased artery. However, the device used is complicated.

There is therefore a need of a means to solve at least one of the problems as discussed above for eliminating the said disease.

### SUMMARY OF THE INVENTION

Accordingly, the present invention in one aspect provides a system for treating a diseased artery, comprising a flexible device for wrapping around the diseased artery, at least an imaging fiber and/or an ablation fiber removably adapted on the inner side of the flexible device for imaging and/or treating the said artery and a controller for controlling the process of imaging or treatment on the said artery.

In another aspect the present invention provides a method for treating a diseased artery, including steps of: approaching the diseased artery from external side, placing and wrapping the flexible device of the system of the present invention over the diseased artery, ablating tunica adventitia and tunica media of the diseased artery up to a fibrous capsule of the atherosclerosis of the diseased artery and exposing atheromatous plaque of the atherosclerosis in the diseased artery and necrotic core to the natural defense system of the body thereby eliminating atherosclerosis.

In a further aspect, the present invention provides a flexible slap-wrap device having predefined outer side and inner side, said inner side has at least one groove extending longitudinally for adapting imaging fiber or laser fiber wherein said flexible device is flat in normal condition and on slapping, forms tube—like structure for wrapping.

According to the present invention, the flexible device has at least one longitudinal groove to adapt the ablation fiber or the imaging fiber.

According to the present invention, the flexible device has at least one window or cut on the longitudinal sides to accommodate side branches of the diseased artery.

### BRIEF DESCRIPTION OF THE INVENTION

A preferred embodiment of the present invention will now be described with reference to the accompanying drawings (which in no way restrict the scope of the invention and are for the purpose of illustration only) in which:

FIG. 1 shows a partially sectioned schematic view of an artery wherein:
FIG. 1A shows a normal artery;
FIG. 1B shows a diseased artery that is artery having thickened wall due to atherosclerosis; and
FIG. 1C shows a partially sectioned schematic view of a widespread and distributed atherosclerosis over a diseased arterial tree;

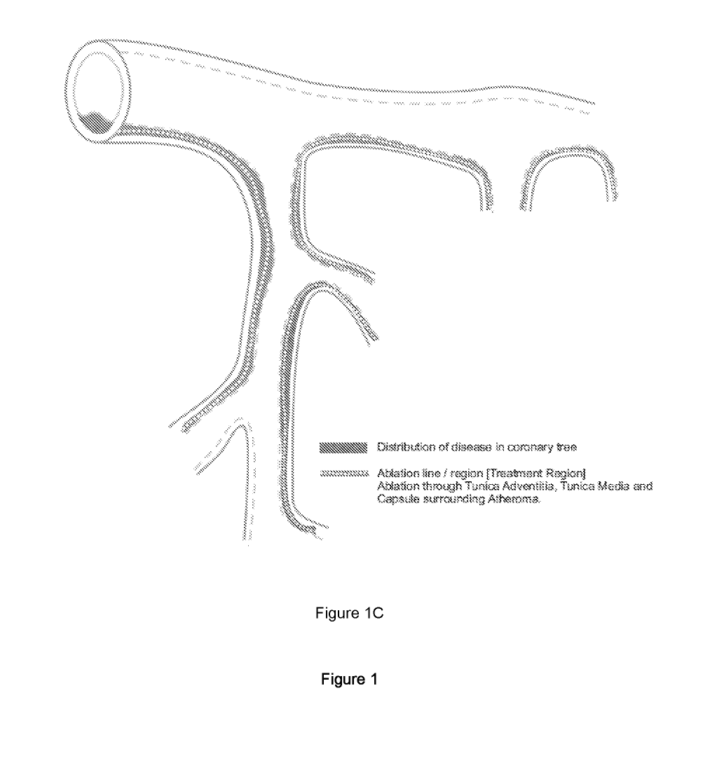

FIG. 2 shows a perspective view of a flexible device over an artery to be treated according to the present invention;

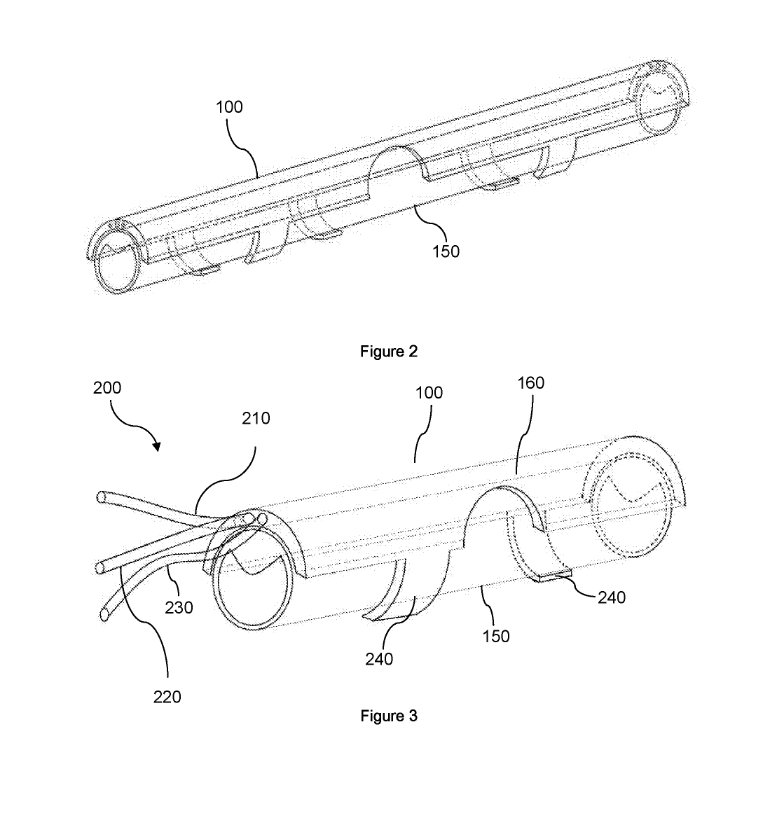

FIG. 3 shows a perspective view of a system according to the present invention;

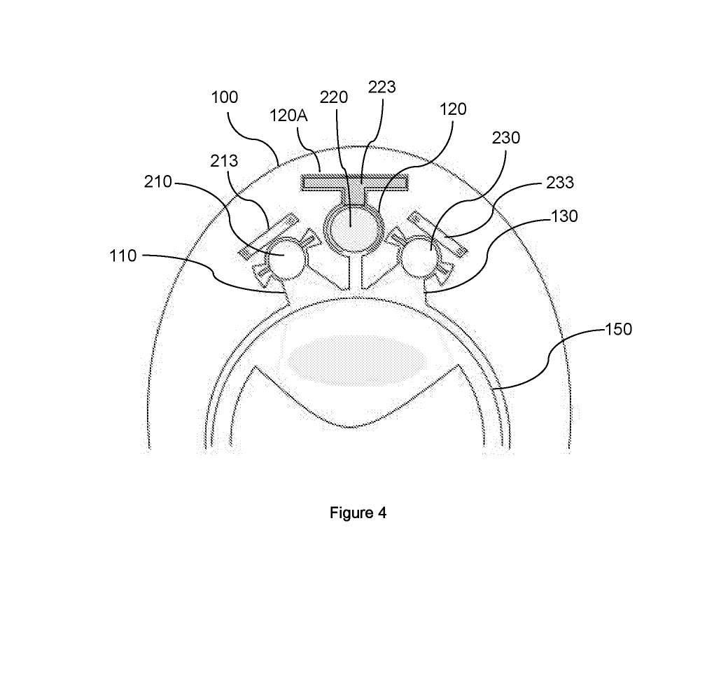

FIG. 4 shows a cross sectional view of the flexible device the present invention;

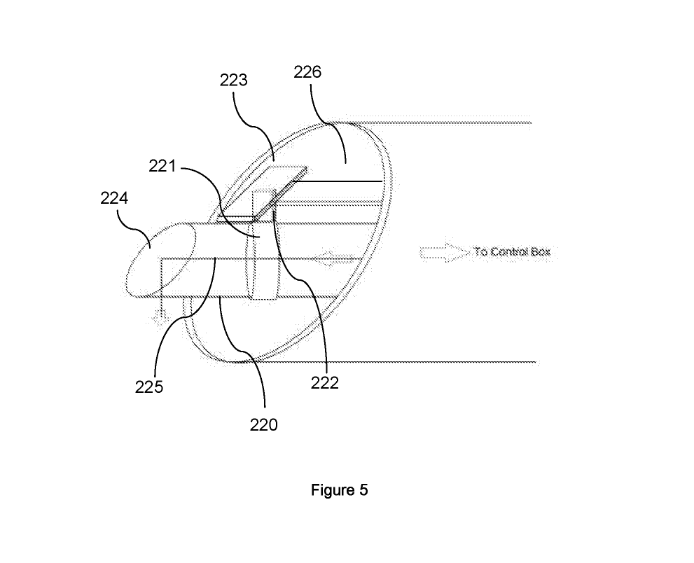

FIG. 5 shows a magnified view of an ablation fiber; and

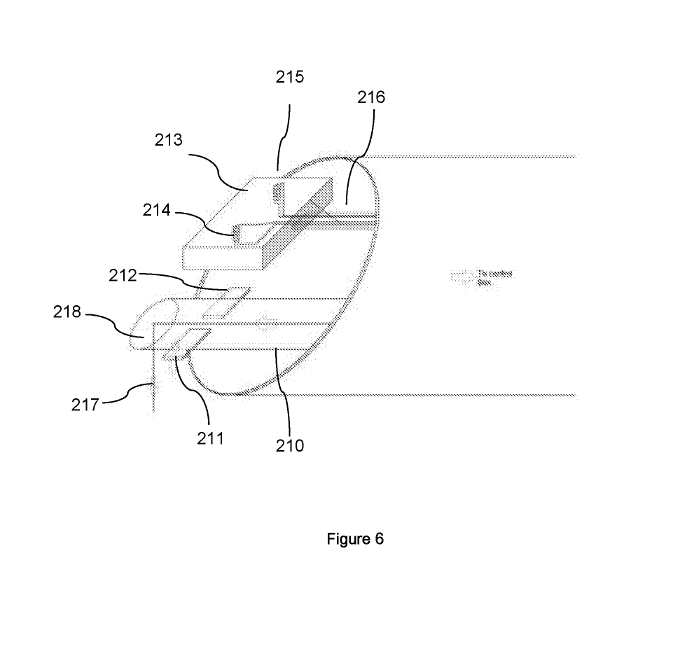

FIG. 6 shows a magnified view of an imaging fiber.

### DETAILED DESCRIPTION OF THE INVENTION

Although specific terms are used in the following description for the sake of clarity, these terms are intended to refer only to the particular structure of the invention selected for illustration in the drawings, and are not intended to define or limit the scope of the invention.

References in the specification to “one embodiment” or “an embodiment” member that a particular feature, structure, characteristics, or function described in connection with the embodiment is included in at least one embodiment of the invention. The appearances of the phrase “in one embodiment” in various places in the specification are not necessarily all referring to the same embodiment.

In general, the present invention in preferred embodiment provides a system for treating a diseased artery having atherosclerosis comprising a flexible device of a slap-wrap type, having inner-side and outer side, at least on of an ablation fiber or an imaging fiber adapted on the inner side of the flexible device for treating the diseased artery and a controller for controlling the process of treatment.

The flexible device according to the present invention is flat in normal condition and wraps the diseased artery to be treated during treatment by forming a tube-like structure on slapping. A person skilled in the art can easily understand that the slapping means touching longitudinal ends or minimum force required to apply on the longitudinal ends of the flexible device to change the shape of the flexible device. The flexible device has one or more arms to hug or wrap the diseased artery to be treated.

According to an embodiment of the invention the present invention, the flexible device has at least one window and/or cut on the longitudinal sides to accommodate side branches of the diseased artery.

According to a preferable embodiment of the present invention, the flexible device has at least one longitudinal groove to adapt either the ablation fiber or the imaging fiber. Alternatively, the flexible device has one groove extending longitudinally on the inner side for adapting the ablation fibre movably to and fro, and an imaging fibre one on top of one-another.

According to the more preferable embodiment of the present invention, the flexible device has two grooves extending longitudinally on the inner side for adapting the ablation fiber movably to and fro and an imaging fiber side by side. Advantageously, the flexible device has three grooves extending longitudinally on the inner side for adapting the ablation fiber movably to and fro in the middle groove and two imaging fibers adapted rotatably in the adjacent groves for taking images of the diseased artery before ablation and during the ablation process in real time.

According to the preferable embodiment of the present invention, the ablating fiber has a laser means for ablating artery from outer side of the diseased artery. Alternatively, the ablating fiber may be a mechanical ablation device including at least one blade mounted on the ablation fiber with pressure means/sensors.

According to an embodiment of the present invention, the imaging fibers can be rotated by a rotating means for covering wide angle of the image.

According to the present invention, the length of the flexible device depends upon the length of the disease spread in the artery. Preferably, the length of the flexible device is more than the length of the disease spread in the artery.

The present invention also provides a method for treating an artery including steps of approaching a diseased artery from external side, wrapping the flexible device by placing and slapping the flexible over the diseased artery, ablating artery wall including ablation of tunica adventitia and tunica media of the diseased artery and a fibrous capsule of the atherosclerosis and exposing atheromatous plaque of the atherosclerosis and necrotic core to the natural defense system of the body thereby eliminating atherosclerosis. The ablating means ablates whole length or to the extent of the diseased artery.

Advantageously, the method comprises a step of surveying thickness of the arterial wall and a length of the atherosclerosis in the diseased artery, prior to ablating step, the diseased artery with the imaging fibers of the flexible device and during ablating step, calculating depth of ablating incision required to expose the atheromatous plaque from the diseased artery in real time.

A most preferable embodiment of the system for treating a diseased artery and the flexible device according to the present invention is described in detail hereinafter with reference to the accompanying drawings for the understanding of the present invention.

FIG. 2 shows a flexible device (100) snapped over the artery (150) for ablation according to the preferred embodiment of the present invention.

FIG. 3 shows a system (200) for treating an artery (150) according to the preferred embodiment of the present invention comprising the flexible device (100), fibers (210, 220, 230) extending from the flexible device (100) towards the controller (not shown) for controlling said fibres (210, 220, 230) inside said flexible device (100).

The flexible device (100) according to the present invention hereinafter referred to as an arterial sheath.

According to the present invention, the arterial sheath (100) can be made of silastic or similar flexible biocompatible polymer. The arterial sheath (100) as shown in Figures is a semi-cylinder with multiple flexible arms (240) on either side of the arterial sheath (100) capable of hugging the artery (150). The arterial sheath (100) of the present invention can be flattened for placing over the artery (150). Upon release/slapping or by applying nominal force, the arterial sheath (100) takes a tube-like structure shape to surround the artery (150) to be treated. Alternatively, the arms (240) can be successively bent and placed around the artery one by one. The arterial sheath (100) has one or more windows or cuts (160) on the longitudinal sides to accommodate side branches (not shown) of the artery (150). Advantageously, said arterial sheath (100) can be made of silastic or similar flexible biocompatible polymer.

As shown in FIG. 3, said imaging fibre (210, 230) is used for taking the image of the said artery (150) to provide a broader view of the said artery (150) to a user. The ablation fiber (220) is used for ablation of said artery (150).

The controller (not shown) makes the ablation fiber (220) move to and fro along the length of the sleeve. The fiber is moved according to the programming instruction from the controller. Similarly, the controller moves the imaging fibers (210, 230) to and fro along the length of the sleeve as well as rotate in the grooves.

The arterial sheath (100) shown in the Figures, has three grooves (110, 120, 130) on the inner side of the arterial sheath (100). Each groove accommodates a fibers (210, 220, 230) for the process to be required. According to the preferred embodiment of the invention and as shown in the FIG. 4, The central groove (120) accommodates an ablation fiber (220) and the adjacent grooves (110, 130) accommodate the imaging fibers (210, 230). Each groove of the arterial sheath (100) is connected to a tubular sheath (not shown) that protect the fibers and control wires and convey the fibers to the controller (not shown).

As shown in FIG. 3, said ablation fiber (220) is capable of transmitting high intensity laser (225). According to the present invention, the laser (225) emitted by the ablation fiber is a Femto-second pulsed laser having pulses preferably between 10 to 750 fs duration and fluence 2 to 20 Joules per square centimeter.

As shown in the FIG. 4, the arterial sheath (110) has a stabilizer groove (120A) extending parallel above the middle groove or the ablation groove (120). The ablation fiber stabilizer (223) extends through the stabilizer groove (120A) and stabilizes the ablation fiber (220) for carrying out ablation of the diseased artery (150).

As shown in FIG. 4, the adjacent grooves (110, 130) are spaced for the accommodating the imaging fiber controller means (213, 233) that allows the imaging fiber (210, 230) to rotate about a longitudinal axis for wide angle.

Referring FIG. 5 shows a magnified view of the ablation fiber (220) described in FIG. 3 and FIG. 4 according to the present invention. As shown in FIG. 5, said ablation fiber (220) has a mirror (224) on a tip. According to the preferred embodiment of the present invention, the mirror (224) is placed at about 45 degrees. The mirror ensures that the ablation laser beam (225) travelling along the length of the fiber is reflected vertically down, onto the surface of the artery (150) to be treated. A collar (221) around the ablation fiber (220) attaches through a vertical bar (222) to the ablation fiber stabilizer (223). This arrangement ensures that the ablation fiber (220) does not rotate within the groove in the sleeve. The ablation fiber stabilizer (223) is connected to the controller (not shown) with a wire (226) so that it can facilitate movement of the ablation fiber (220), back and forth, along the length of the sleeve.

Referring FIG. 6 shows a magnified view of said imaging fiber (210) as described in FIG. 3 and FIG. 4 according to the present invention. Each imaging fiber (210) also has a mirror (218) on the tip. According to the preferred embodiment of the present invention, the mirror is placed at 45 degrees.

As shown in FIG. 6, each imaging fiber (210) is stabilized in the groove by two wings (211, 212) placed along the transverse axis of imaging fiber (210). These wings (211, 212) are adapted fixedly/removably to the optical fiber (210). Above the imaging fiber (210) is a space for the imaging fiber controlling means (213). The controlling means (213) contains two magnetic coils (214, 215). These coils (214, 215) are connected through wires (216) to the controller (not shown). Low voltage direct current runs through the coils to magnetize as per the requirements of the rotation required for imaging the artery (150). The current magnetizes the coils (214, 215) which attract the wings (211, 212) attached to the sides of the imaging optical fiber (210) that makes the imaging optical fiber to rotate in an arc, thereby scanning the imaging beam (217) across the artery (150). This makes possible to take Optical Coherence Tomography of the artery wall.

For treating the diseased artery, according to the method of the present invention the diseased artery is approached from outside and the flexible device of the present invention is kept over the artery to be treated and wrapped around the artery. Then survey of the diseased artery is required to be carried out for calculating thickness of the arterial wall and a length of the atherosclerosis in the diseased artery, prior to ablating step. Accordingly, the controller moves the imaging fibers to and fro along the length of the sleeve as well as rotate in the grooves to take images of artery before the ablation to calculate the length and depth of the ablation wherein depth of the ablation include thicknesses of tunica adventitia and tunica media of the diseased artery and fibrous capsule of the atherosclerosis of the diseased artery.

Once the thickness to be ablated is calculated, the method of the present invention includes step of ablating artery wall including ablation of tunica adventitia and tunica media of the diseased artery and a fibrous capsule of the atherosclerosis and exposing atheromatous plaque of the atherosclerosis and necrotic core to the natural defense system of the body thereby eliminating atherosclerosis. Accordingly, the controller makes the ablation fiber move to and fro along the length of the sleeve for ablating the diseased artery along the length of the disease of the artery or to an extent of the artery. The fiber is moved according to the programming instruction from the controller and ablates whole length or to the extent of the diseased artery. On ablation/incision, the contents plaques are exposed to the natural defense of the body and are destroyed by the natural defense system. The plaque escaping out of the artery on the external surface of the artery may be wiped out during or after the ablation procedure. Then, natural healing of the artery is allowed which eliminates atherosclerosis thoroughly. The ablation is carried out in such a way that the innermost layer of the artery namely tunica intima remains undisturbed.

The nouns inner side, outer side etc. used in the specification for understanding purpose and are not limiting the invention to a meaning of said nouns. The foregoing description of the invention has been set merely to illustrate the invention and is not intended to be limiting. Since modifications of the disclosed embodiments incorporating the substance of the invention may occur to a person skilled in the art, the invention should be construed to include everything within the scope thereof as claimed in the following claims:

1. A system for treating a diseased artery, comprising:
a flexible device for wrapping around the diseased artery;
at least an imaging fiber and/or an ablation fiber removably adapted on inner side of the flexible device for imaging and/or treating the said artery and
a controller for controlling the process of imaging or treatment on the said artery.

2. The system for treating a diseased artery as claimed in claim 1, wherein said flexible device is flat in normal condition and wraps the diseased artery to be treated during treatment.

3. The system for a treating diseased artery as claimed in claim 1, wherein the said flexible device has one or more arms to hug or wrap the diseased artery to be treated.

4. The system for a treating diseased artery as claimed in claim 1, wherein the flexible device has at least one window or cut on the longitudinal sides to accommodate side branches of the diseased artery.

5. The system for treating a diseased artery as claimed in claim 1, wherein the flexible device has at least one longitudinal groove to adapt the ablation fiber or the imaging fiber.

6. The system for treating a diseased artery as claimed in claim 1 or 5, wherein the flexible device has one groove extending longitudinally on the inner side for adapting the ablation fiber movably to and fro and an imaging fiber one on top of one-another.

7. The system for treating a diseased artery as claimed in claim 1 or 5, wherein the flexible device has two grooves extending longitudinally on the inner side for adapting the ablation fiber movably to and fro and an imaging fiber side by side.

8. The system for treating a diseased artery as claimed in claim 1, wherein the flexible device has three grooves extending longitudinally on the inner side for adapting the ablation fiber movably to and fro in the middle groove and two imaging fibers adapted rotatably in the adjacent groves for taking images of the diseased artery.

9. The system for treating a diseased artery as claimed in claim 1, wherein the ablating fiber has a laser means for ablating artery from outer side of the diseased artery.

10. The system for treating a diseased artery as claimed in claim 1, wherein the ablating fiber is a mechanical ablation device including at least one blade mounted on the ablation fiber with pressure means.

11. The system for treating a diseased artery as claimed in claim 1, wherein the imaging fiber is adapted with a rotating means for covering wide angle of the image.

12. A method for treating a diseased artery, including steps of:
approaching the diseased artery from external side;
wrapping the flexible device of the system as claimed in claim 1 by placing and slapping over the diseased artery;
ablating artery wall extending from tunica adventitia and tunica media of the diseased artery up to a fibrous capsule of the atherosclerosis of the diseased artery; and
exposing atheromatous plaque of the atherosclerosis and necrotic core to the natural defense system of the body thereby eliminating atherosclerosis.

13. The method for treating a diseased artery as claimed in claim 12, comprises a step of surveying thickness of the arterial wall and a length of the atherosclerosis in the diseased artery with the imaging fibers of the flexible device prior to ablating the diseased artery and during the ablating step, calculating depth of ablating incision required to expose the atheromatous plaque from the diseased artery in real time.

14. A flexible slap-wrap device having predefined outer side and inner side, said inner side has at least one groove extending longitudinally for adapting imaging fiber or laser fiber wherein said flexible device is flat in normal condition and on slapping, forms tube-like structure for wrapping.
